(19) United States
(12) Patent Application Publication
(10) Pub. No.: US 2006/0241641 A1
(43) Pub. Date: Oct. 26, 2006

(54) Methods and instrumentation for distraction and insertion of implants in a spinal disc space

(75) Inventors: William J. Albans, Cordova, TN (US); Jason Eckhardt, Memphis, TN (US)

Correspondence Address: KRIEG DEVAULT LLP, ONE INDIANA SQUARE, SUITE 2800, INDIANAPOLIS, IN, 46204-2709, US

(73) Assignee: SDGI Holdings, Inc.

(21) Appl. No.: 11/112,586

(22) Filed: Apr. 22, 2005

(51) Int. Cl. A61B 17/58

(52) U.S. Cl. 606/90

(57) ABSTRACT

Instruments for inserting an artificial disc implant in a space between adjacent bony portions include upper and lower guide members separated by a spreader with an implant positioned forwardly of the spreader. The spreader is movable forwardly between the guide members with a drive member to position the implant in a space between the bony portions while engaging at least a portion of the implant with at least one vertebra. The spreader contacts the adjacent bony portions to facilitate withdrawal of the inserter instrument when the implant is positioned in the space and engaged with the at least one vertebra.

### BACKGROUND

The repair and reconstruction of bony structures is sometimes accomplished by directly fixing adjacent bony portions to each other, such as by a plate. In other instances, bone growth inducing material can be introduced between the adjacent bony portions, which over time results in a solid bony connection. In some instances, the adjacent bony portions are not sufficiently strong to maintain their patency as the bone heals or the bone grows between the adjacent structures through the bone growth inducing material. In these instances, grafts, cages, artificial joints and other implants have been provided to engage the adjacent bony structures to provide additional stability.

One problem, among others, with such implants is associated with positioning the implant in the space between adjacent bony portions. Insertion can be difficult or time consuming if the bony portions are spaced too close together, or if the adjacent tissue, nerves or vasculature impedes access to or placement of the implant in the space between the bony portions. Furthermore, maintenance of distraction of the space during insertion of the implant requires additional instruments in the space or in the operative approach to the space which can make the procedure more invasive and impede access and visibility during implant insertion.

### SUMMARY

The invention provides instruments that facilitate placement of an artificial disc and other implants and instruments between adjacent vertebrae of a spinal column.

According to one aspect, there is provided an instrument for positioning an implant in a space between adjacent vertebrae. The instrument includes a housing and a pair of opposing guide members coupled to the housing. Each of the guide members includes a body with an outer surface and an opposite guide surface and an elongated slot opening therebetween. The slot extends along and opens at a distal end of the respective guide member. The distal ends of the guide members are positionable in the space between vertebrae. The instrument also includes a spreader positioned between the pair of guide members. The spreader includes a central body and with a pair of oppositely extending wings. Each wing includes a body slidingly received in a slot of a corresponding one of the pair of guide members and an enlarged outer end. A drive member is coupled to the spreader and operable to forwardly advance the spreader toward the distal ends of the guide members.

In another aspect, there is provided a system for stabilizing a spinal disc space while preserving motion capabilities of the vertebrae adjacent the disc space. The system includes a pair of opposing guide members extending from a handle assembly. Each of the pair of guide members includes a body with an outer surface and an opposite guide surface and a spreader positioned between the pair of guide members. An artificial disc implant includes upper and lower plate members and with an articulating member therebetween. The implant is positionable between the guide surfaces forwardly of and in engagement with the spreader with at least one anchoring member of the implant extending through at least one of the guide members. The system also includes a drive member coupled to the spreader and operable to forwardly advance the spreader and the artificially disc implant toward distal ends of the guide members.

According to a further aspect, a method for inserting an implant in a space between adjacent vertebrae is provided. The method employs an instrument having a housing and a pair of opposing guide members coupled to the housing. The guide members each include an elongate central slot extending therealong and opening at a distal end of the guide member. The instrument also includes a spreader positioned between the pair of guide members and a drive member coupled to the spreader and extending though the housing. The method includes: providing an implant inserter; pivoting at least one of the pair of guide members away from the other of the pair of guide members; positioning an implant on the other of the pair of guide members and forwardly of the spreader, the implant including a first anchoring member extending through the slot of the other guide member; and pivoting the at least one guide member toward the other guide member to receive a second anchoring member of the implant through the slot of the at least one guide member.

These and other aspects can be discerned from the following written description and accompanying figures.

### DESCRIPTION OF THE FIGURES

FIG. 1 is a perspective view of an inserter instrument and implant positioned in the inserter instrument.

FIG. 2 is an elevation view of a portion of the inserter instrument of FIG. 1 and the implant.

FIG. 3 is a perspective view of the housing and drive member of the inserter instrument.

FIG. 4 is a perspective view of the housing and drive member assembled with a spreader coupled to a distal end of the drive member.

FIG. 5 is a front elevational view of the spreader.

FIG. 6 is a perspective view of the housing, drive member, and spreader assembled with a lower guide member.

FIG. 7 is the view of FIG. 6 with the spreader moved distally along the guide member.

FIG. 8 is a perspective view of the housing, drive member, spreader and lower guide member assembled with the implant holder and an upper guide member mounted to the housing and pivoted away from the spreader.

FIG. 9 is a perspective view of the assembly of FIG. 8 with an implant positioned forwardly of the spreader on the lower guide member and the upper guide member pivoted adjacent the spreader.

FIG. 10 is an elevation view of a distal portion of the inserter instrument with the implant positioned in a disc space between vertebrae.

FIG. 11 is the elevation view of FIG. 10 with the spreader positioned against the vertebrae to withdraw the guide members from the disc space.

FIG. 12 is a plan view of a cutting instrument movable along at least one of the guide members to prepare a vertebra to receive the implant.

FIG. 13 is an elevation view of the cutting instrument.

FIG. 14 is a section view of the cutting instrument on a guide member of the inserter instrument.

FIG. 15 is an elevation view showing the cutting member on the upper guide member being moved toward the vertebra.

FIG. 16 is an elevation view showing various attachment arrangements for maintaining a positioning of the inserter instrument relative to the vertebrae.

### DESCRIPTION OF THE ILLUSTRATED EMBODIMENTS

For the purpose of promoting an understanding of the principles of the invention, reference will now be made to the illustrated embodiments thereof and specific language will be used to describe the same. It will nevertheless be understood that no limitation of the scope of the invention is thereby intended. Any such alterations and further modifications in the invention, and any such further applications of the principles of the invention as described herein are contemplated as would normally occur to one skilled in the art to which the invention relates.

An instrument is provided for inserting artificial disc and other implants into a space between adjacent bony portions to support the adjacent bony portions. The inserter instrument can be used with any type of bone support implant, such as artificial joints, spacer devices, and fusion devices, for example. The implants can be made from bone material or any suitable biocompatible metal, plastic, or other material. The implants can also be made from combinations of materials, and include multiple components fixed to or movable relative to one another. In one application, the inserter instrument is employed in spinal surgical procedures for inserting an artificial implant in the disc space between adjacent vertebrae. The inserter instrument can also be employed to guide instruments that prepare the vertebrae to receive the implant.

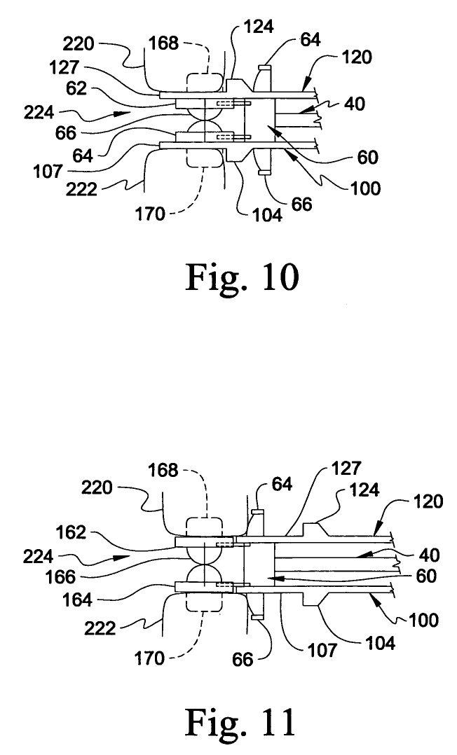

For example, in the illustrated embodiments of FIGS. 10-11, the adjacent bony portions include first vertebra 220 and second vertebra 222. The vertebrae 220, 222 include a disc space 224 therebetween, which provides a space for insertion of an implant between the adjacent bony portions. The inserter instrument can be used in various approaches to the disc space in spinal surgical procedures, including posterior, posterior lateral, transforaminal, lateral, anterior lateral, oblique, and anterior approaches. The inserter can also be used in approaches to various regions of the spine, including the lumbar, thoracic and cervical regions. It is contemplated that the inserter instrument can have application in surgical procedures other than spinal surgical procedures to facilitate insertion of an implant between adjacent bony portions.

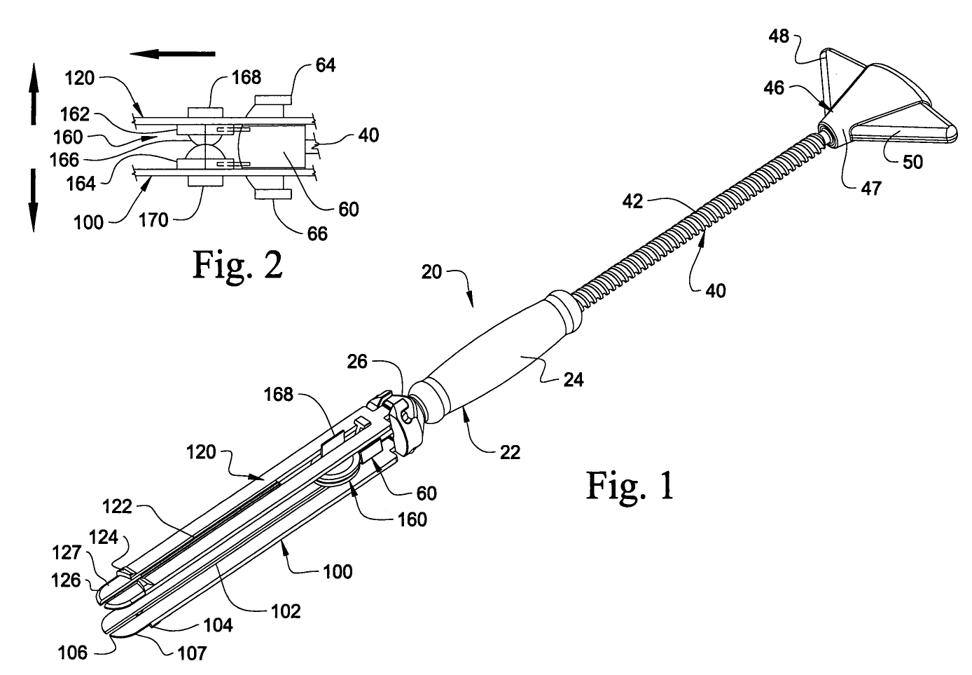

In FIG. 1 an inserter instrument 20 is shown. Inserter instrument 20 includes an intermediate housing 22 with a drive member 40 extending through and coupled thereto. Guide members 100, 120 are coupled to housing 22 and extend distally therefrom. The distal end of drive member 40 extends in the space between guide members 100, 120. The distal end of drive member 40 engages a spreader 60 positioned between guide members 100, 120. An implant 160 is positioned forwardly of spreader 60. Implant 160 can be engaged by spreader 60 to facilitate in maintaining the positioning of implant 160 between guide members 100, 120. Spreader 60 is moveable distally or forwardly by manipulating drive member 40 relative to housing 22 to advance drive member 40 forwardly towards distal ends of guide members 100, 120.

The proximal ends of guide members 100, 120 are pivotally attached to housing 22, facilitating loading of the implant 160 and placement of the distal ends of guide members 100, 120 adjacent one another for positioning in the spinal disc space. As indicated in FIG. 2, as spreader 60 pushes implant 160 distally between guide members 100, 120, the distal ends of guide members 100, 120 can separate and thus apply a distraction force to the adjacent vertebrae. The vertebrae are distracted sufficiently to receive implant 160 since the final distraction height is determined at least by the height of implant 160 between the distal ends of guide members 100, 120.

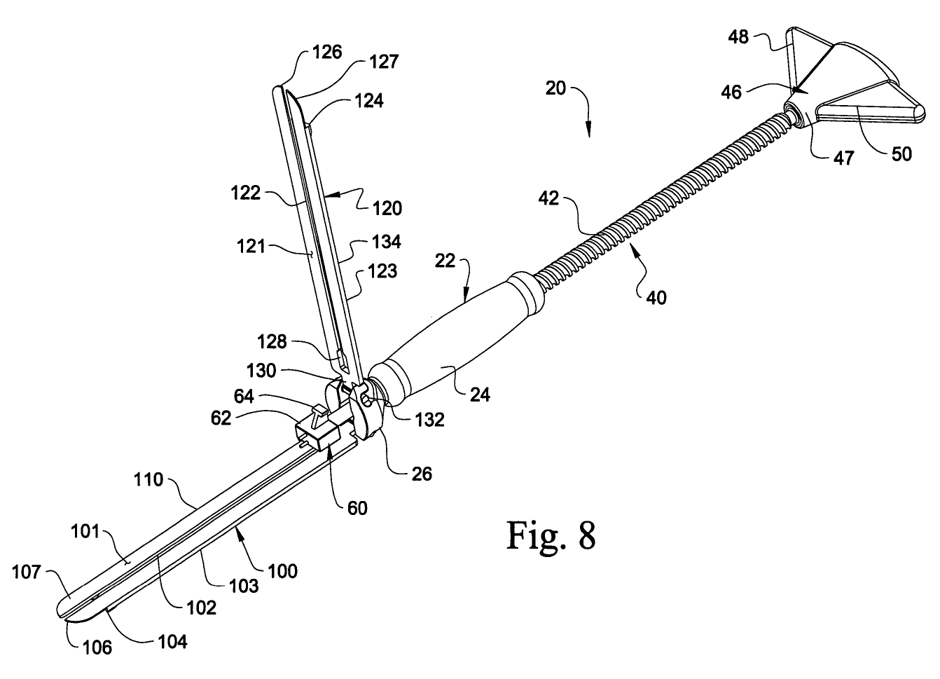

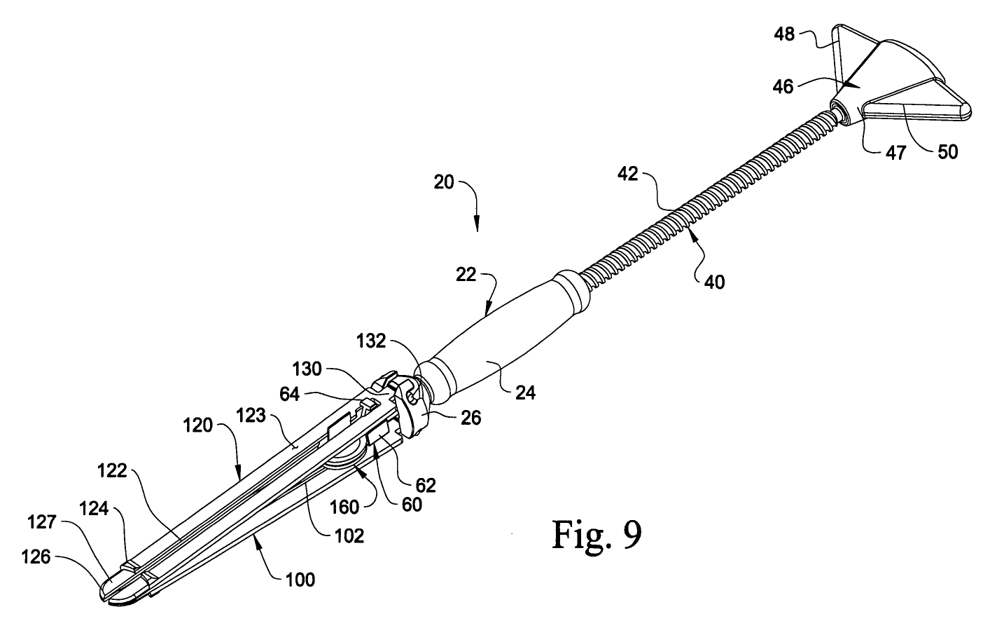

Further details of the assembly of inserter instrument 20 will be discussed with further reference to FIGS. 2-9. Housing 22 includes a drive member engaging portion 24 extending proximally from a distal coupling portion 26. A passage extends through each of drive member engaging portion 24 and coupling portion 26. Coupling portion 26 includes a pair of upwardly and proximally extending upper fingers 28, 32 projecting therefrom. Fingers 28, 32 form proximally opening receptacles 30, 34, respectively, which receive and pivotally capture a guide member 120 (FIG. 8) to coupling portion 26. A vertical slot extends between fingers 28, 32 and along the distally oriented face of coupling portion 26 between a pair of opposite lower fingers (not shown) that are identical to fingers 28, 32. The lower fingers pivotally capture lower guide member 100 to coupling portion 26.

It should be understood that the terms “upper” and “lower” refer to the orientation of the elements of the instruments in the Figures as shown in an operative approach to the space between adjacent bony portions. The instruments can be rotated or repositioned such that, for example, the lower fingers extend upwardly and guide member 100 is positioned above guide member 120.

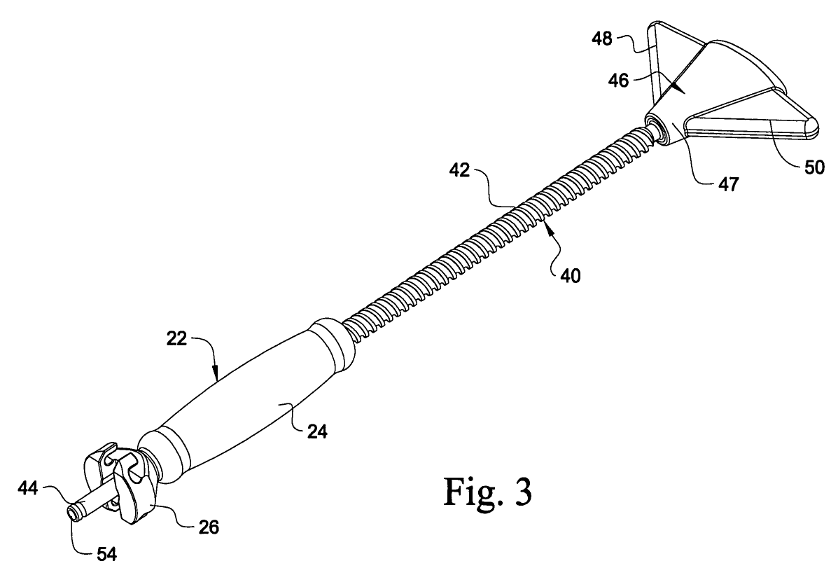

In FIG. 3 drive member 40 is coupled to housing 22. In the illustrated embodiment, drive member 40 includes a shaft 42 and proximal handle 46. Handle 46 includes opposite first and second arms 48, 50 extending from a central body portion 47 to facilitate grasping of handle 46. Shaft 42 extends through a passage of housing member 22 to a distal end 44. At least a portion of shaft 42 is threaded to threadingly engage an internally threaded portion of housing member 22. Accordingly, drive member 40 is movable longitudinally distally and proximally by rotating shaft 42 in housing 22, thereby distally or proximally displacing distal end 44.

In another embodiment, drive member 40 can include a ratchet mechanism. A ratchet bar can be provided along shaft 42, which is moved linearly in housing 22 to distally advance spreader 60 between guide members 100, 120. A handheld pistol grip-like handle with a trigger, a threaded screw with a thumbwheel thereabout, or other suitable handle can be provided to effect the linear movement of the ratchet bar. A catch mechanism, either in the handle or housing, can maintain the distal positioning of the ratchet bar until it is released, allowing the ratchet bar to be linearly and proximally moved by actuating the trigger mechanism.

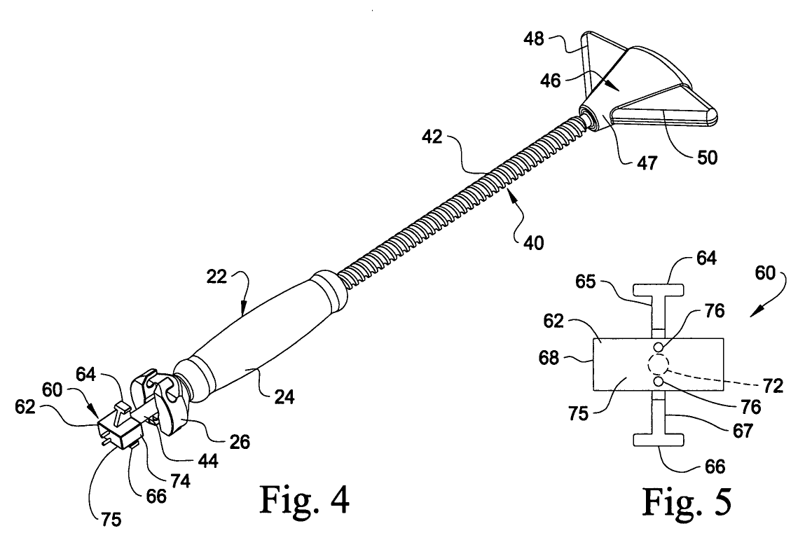

In FIG. 4 there is shown spreader 60 attached to distal end 44 of drive member 40. Spreader 60 includes a central body 62 having an upper wing 64 and a lower wing 66 extending therefrom in opposite directions from one another, as shown in FIG. 5. A bore 72 extends centrally through central body 62, and can open at the proximal end 74 and distal end 75 thereof. Bore 72 can also be a blind hole opening proximally. Distal end 44 of drive member 40 is rotatably received in the trailing or proximal end opening of bore 72. In the illustrated embodiment, distal end 44 includes a circumferential groove to receive a ball plunger in spreader 60. Other suitable rotatable coupling arrangements are also contemplated, such as a C-ring, an expandable distal end 44, or a distal end 44 positionable about and engageable to an extension or post extending proximally from spreader 60. Depending on the direction of rotation of drive member 40 about its longitudinal axis, spreader 60 moves distally or proximally without rotation.

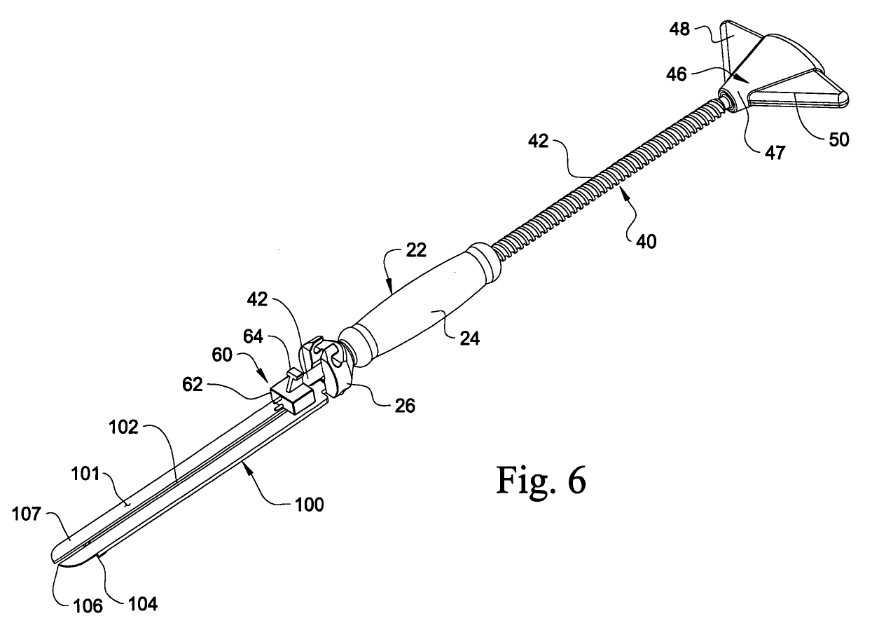

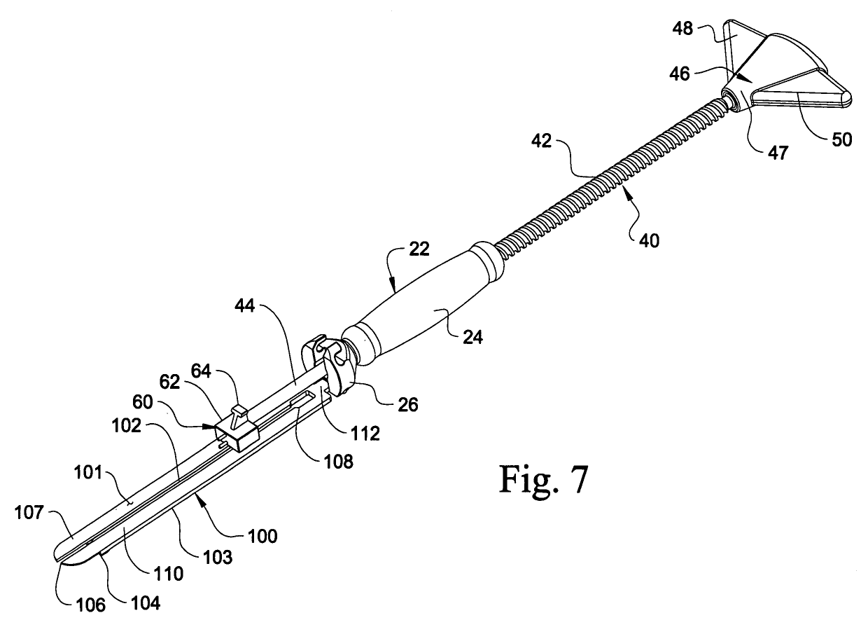

Spreader 60 is movable with drive member 40 from a proximal position shown in FIG. 6 to a more distal location relative to guide members 100, 120, as shown in FIG. 7. Spreader 60 further includes leading or distal end wall 75 that includes one or more engaging members 76. Engaging members 76 are engageable to implant 160 positioned forwardly thereof, and resist rotation of the implant 160 relative to spreader 60 as it is advanced between guide members 100, 120. Engaging members 76 may be in the form of pins that are received in frictional engagement in aligned bores on the implant 160. Other embodiments contemplated other forms for engaging members 76 and other engagement relations between implant 160 and the engaging members. For example, one or more engaging members may be provided at a distal end of an implant holder extending through drive member 40 and spreader 60 that is remotely operable to engage and release an implant forwardly of spreader 60.

Referring to FIG. 6, lower guide member 100 is pivotally coupled to coupling portion 26 of housing 22. Upper guide member 120 can be pivotally coupled with the upper fingers 28, 32 of coupling portion 26, as shown in FIG. 8. The proximal ends of guide members 100, 120 include laterally oriented crossbars, such as crossbar 132 shown in FIG. 8. The ends of crossbar 132 are received in corresponding ones of the proximally opening receptacles 30, 34, and reside against the fingers 28, 32, which maintain guide member 120 in pivotal and removable engagement with housing 22. Lower guide member 100 is similarly pivotally and removably coupled to the opposite, downwardly extending fingers of coupling portion 26.

The ability to quickly disassemble guide members 100, 120 allows inserter instrument 20 to be cleaned and sterilized after the surgical procedure is completed. It further allows guide members 100, 120 to be provided in a set of guide members for use with a common housing, drive member and implant holder. For example, the guide members in the set can include various lengths, widths, or abutment member configurations from which the surgeon may select during surgery. Other embodiments contemplated guide members that are not removably coupled to housing 26, or guide members that are not easily removed for cleaning purposes.

Guide members 100, 120 extend distally from housing 22, and define a path for insertion of an implant between the adjacent bony portions, such as vertebrae 220, 222. As shown in FIGS. 7-8, guide member 100 includes a body 110 extending from a proximal end 112 to a distal end 106. Body 110 can be provided with an elongated guide slot 102 extending therethrough along a central axis of body 110. Guide slot 102 opens along a guide surface 101 and an opposite outer surface 103 of body 110. Guide slot 102 extends from a location adjacent proximal end 112 through distal end 106. Guide slot 102 includes an enlarged proximal end opening 108 for passage of the enlarged outer end of lower wing 66. The remaining proximal portion of guide slot 102 is sized to slidingly receive the body 67 of lower wing 66, but prevents passage of the enlarged outer end of wing 66 therethrough.

Similarly, guide member 120 includes a body 134 extending from a proximal end 130 to a distal end 126. Body 134 can be provided with an elongated guide slot 122 extending therethrough along a central axis of body 134. Guide slot 122 opens along a guide surface 121 and opposite outer surface 123 of body 134. Guide slot 122 extends from a location adjacent proximal end 130 to a location through distal end 126. Guide slot 122 includes an enlarged proximal end opening 128 for passage of the enlarged outer end of upper wing 64. The remaining proximal portion of guide slot 122 is sized to slidingly receive the body 65 of upper wing 64, but prevents passage of the enlarged outer end of wing 64 therethrough.

Guide member 100 can be provided with an abutment member 104 adjacent distal end 106 projecting from outer surface 103 for contacting the adjacent bony structure to limit the insertion depth of guide member 100 into the space between the adjacent bony portions. A support portion 107 of guide member 100 extends distally from abutment member 104 and into the space between the adjacent bony portions, forming an extension of and being co-planar with guide surface 101 and outer surface 103. Guide member 120 can be provided with an abutment member 124 projecting from outer surface 123 adjacent distal end 126 for contacting the adjacent bony portion to limit the insertion depth of guide member 120 into the space between the adjacent bony portions. A support portion 127 extends distally from abutment member 124 and into the space between the adjacent bony portions, forming an extension of and being co-planar with guide surface 121 and outer surface 123.

When assembled to housing 22, the guide surfaces 101, 121 of guide members 100, 120 are oriented toward one another. Support portions 107, 127 can extend along an adjacent surface of the adjacent bony portion to facilitate insertion of the implant 160 into the space between the adjacent bony portions. Support portions 107, 127 also contact the adjacent bony portions to distribute a spreading or distraction force thereto. The spreading or distraction force can be applied to the adjacent bony portions by separating guide members 100, 120 as the implant 160 and spreader 60 are distally advanced between guide members 100, 120. Support portions 107, 127 further protect the adjacent vertebral endplate as implant 160 is positioned in the space between the adjacent bony portions, and facilitate insertion of implant 160 in the desired position in the space between the adjacent bony portions.

In one embodiment, it is contemplated that implant 160 is selected from a set of implants having various heights and or angulation between its upper and lower surfaces. The implant of the appropriate height can be selected to provide a height that corresponds to a desired restored disc space height when implanted. If vertebral motion is desired, implant 160 can be an artificial disc implant. In the illustrated embodiment of FIG. 2, implant 160 includes an upper plate 162, a lower plate 164, and an articulating member 166 therebetween. Articulating member 166 is movable to maintain spinal motion. Articulating member 166 may be in the form of a ball and socket joint, compressible cushioning member, one or more springs, or other device or substance that allows at least limited motion between the adjacent vertebrae 220, 222.

Implant 160 further includes an upper anchoring member 168 extending from upper plate 162, and a lower anchoring member 170 extending from lower plate 164. Anchoring members 168, 170 are engageable to a respective adjacent vertebra to maintain a positioning of implant 160 in the disc space 224. Anchoring members 168, 170 may be in the form of elongated blades that are alignable along and positionable through the adjacent slots 122, 102 of guide members 120, 100. Anchoring members 168, 170 are movable along guide members 120, 100 and into engagement with the adjacent vertebrae as implant 160 is positioned in disc space 160.

Implant 160 is positionable between guide members 100, 120 when one of the guide members is moved away from the other, as shown in FIG. 8. Spreader 60 is positioned with wing 66 aligned with proximal end opening 108 of slot 102. Implant 160 can then be positioned forwardly or distally of spreader 60 with anchoring member 170 through slot 102. The pivoting coupling arrangement of guide members 100, 120 allows distal ends 106, 126 to be positioned adjacent one another so that upper wing 64 is received through end opening 130 of slot 122 and anchoring member 168 is received though slot 122. Implant 160 is then captured between guide members 120, 100 with bodies 65, 67 extending through respective ones of the slots 122, 102 and guide surfaces 121, 102 in contact with the outer surface of respective ones of the upper and lower plates 162, 164.

Distal ends 106, 126 of guide members 100, 120 can be positioned adjacent one another when implant 160 and spreader 60 are adjacent the proximal ends of guide members 100, 120. This provides a low profile arrangement that allows positioning of support portions 107, 127 in the disc space 224, even if the disc space has collapsed due to removal of distraction. Abutment members 104, 124 are positionable in contact with the adjacent vertebral bodies, preventing over insertion of guide members 100, 120 into the disc space. In the illustrated embodiment, abutment members 104, 124 are orthogonally oriented to the central axis of the guide members, aligning guide members 100, 120 for implant insertion approach along or parallel to, for example, the sagittal plane in an anterior approaches to spinal procedures. In another embodiment, abutment members are obliquely oriented to the central longitudinal axis of guide members 104, 124 to facilitate placement of guide members 100, 120 in an approach obliquely oriented to, for example, the sagittal plane in spinal procedures.

Implant 160 is advanced distally between guide members 100, 120 by rotating drive member 40 within housing 22, distally advancing drive member 40 and thus spreader 60 and implant 160 along guide members 100, 120. As implant 160 and spreader 60 are distally advanced, the guide members 100, 120 are spread apart or separated from one another. This separation causes support portions 107, 127 to separate and exert a distraction force on the vertebral endplates, separating vertebrae 220, 222 a sufficient distance to accommodate implant 160 therebetween.

In FIG. 10 implant 160 is positioned in the spinal disc space 224. Wings 64, 66 are aligned with abutment members 124, 104 adjacent the vertebrae 220, 222, respectively. Support portions 127, 107 are positioned between the inserted implant 160 and the adjacent vertebral endplate, making manual withdrawal of inserter 20 from the disc space difficult. Wings 64, 66 each include a distal end wall along bodies 65, 67 that extends from body 62 of spreader 60 to the enlarged outer end of the respective wing 64, 66.

Slots 102, 122 extend through abutment members 104, 124, providing an avenue for further advancement of spreader 60 and implant 160 relative to guide members 100, 120. Drive member 40 can be rotated to position wings 64, 66 in contact with vertebrae 220, 222. As driver member 40 is further manipulated, wings 64, 66 and act on the vertebrae to completely displace spreader 60 to distal ends of slots 102, 122, as shown in FIG. 11. When wings 64, 66 contact the adjacent vertebrae 220, 222, spreader 60 does not advance further toward the disc space. Wings 64, 66 instead act on vertebrae 220, 222 to displace guide members 100, 120 proximally, withdrawing support portions 107, 127 from the space between implant 160 and the adjacent vertebral endplates. The distal end opening of slots 102, 122 allows anchoring members 168, 170 to pass therethrough. This allows inserter instrument 20 to be readily withdrawn from the operative site without twisting or impaction, which could disrupt implant positioning in the disc space.

As implant 160 is guided between guide members 100, 120 into the space between the adjacent bony portions, the positioning of implant 160 is controlled in the cephalad/caudal directions by contact of guide surfaces 101, 121 with implant 160. Guide surfaces 101, 121 align implant 160 with the space between the adjacent bony portions. The lateral positioning of implant 160 along guide members 100, 120 is controlled by engagement of implant 160 with engaging members 76 so that implant 160 does not slip out from between guide members 100, 120, where it might contact or damage tissue, nerves, vasculature or other tissue structures adjacent the bony portions on the approach to the space therebetween. Anchoring members 168, 170 extending through slots 122, 102 also maintaining implant 106 between the guide members. Anchoring members 168, 170 are driven into the respective vertebrae 220, 222 to secure implant 160 in position between vertebrae 220, 222.

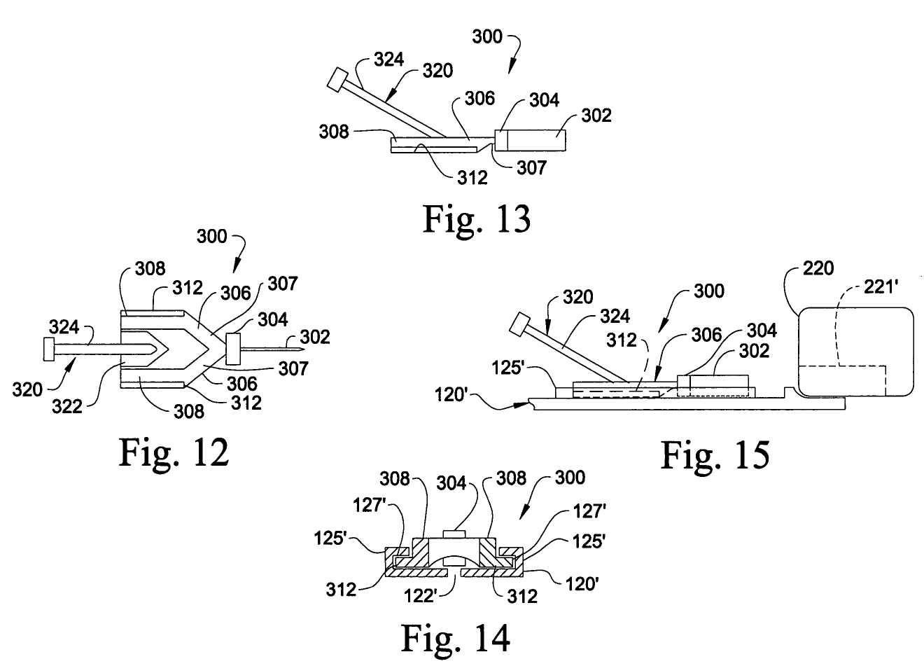

Inserter instrument 20 can also be employed as a guide to guide placement of instruments to prepare vertebrae 220, 222 to receive implant 160. One example of a preparation instrument is a chisel 300 shown in FIGS. 12-15. Various preparation instruments are contemplated that could be guided with inserter instrument 20, including center-cut chisels, comer cut chisels, distractors, rasps, scrapers, and reamers, for example.

Chisel 300 is a center cut chisel with a distal cutting member 302 and an abutment member 304 at a distal end thereof. Chisel 300 further includes proximally extending legs 306 extending from abutment member 304. Legs 306 include angled portions 307 in a diverging relation to one another and parallel portions 308 extending proximally from angled portions 307. An impactor 320 is positionable between parallel portions 308, and movable into contact with the junction of angled portions 307 to apply impaction forces to chisel 300.

Impactor 320 includes an impactor head 322 and a shaft 324 to facilitate transfer of the impaction forces. Impactor head 322 can include a distally tapered distal end to fit between angled portions 307 and a proximal portion slidably received between parallel portions 308.

In use, chisel 300 is positioned on and guided on the guide members, such as guide member 120′. Guide member 120′ is similar to guide member 120, but includes rails 125′ extending from the lateral sides thereof that form inwardly facing slots 127′. Rails 125′ extend along a portion of the length of guide member 120′, and allow chisel 300 to be end loaded into rails 125′ along the outer surface of guide member 120′. Other embodiments contemplate other interfitting arrangements between the chisel and guide member, such as a slot or groove formed in any portion of the guide member that can receive a portion of the chisel.

The vertebrae can be distracted by advancing a spreader without wings or an implant positioned forwardly thereof to a location adjacent support portions 107, 127. Parallel portions 308 of legs 306 include extensions 312 that are positioned in respective ones of the slots 127′ formed by rails 125′ of guide member 120′. This dovetail arrangement slidably secures chisel 300 on guide member 120′ with blade 302 aligned with slot 122′. Rails 125′ further guide chisel 300 distally along guide member 120′ to insert blade 302 into vertebra 220 along a path aligned with slot 122′. Impactor 320 may be employed as needed to deliver impaction forces to facilitate forming a chisel cut 221′ in the bone of the vertebral body, as shown in FIG. 15. Abutment member 304 extends laterally outwardly from blade 302 and limits the depth into which blade 302 can be driven into the vertebral body. The procedure can then be repeated for the other vertebra 222.

After forming the chisel cuts in vertebrae 220, 222, implant 160 can be loaded between the guide members with anchoring members 168, 170 extending through slots of the guide member as discussed above. The guide members 100, 120 are aligned with the disc space, and implant 160 is guided along guide members to position implant 160 between the vertebrae with anchoring members 168, 170 received in the previously formed chisel cuts. The guide members can then be withdrawn proximally by passing the distal end opening of each of the guide members proximally along the respective anchoring members 168, 170 engaged to the vertebrae 220, 222.

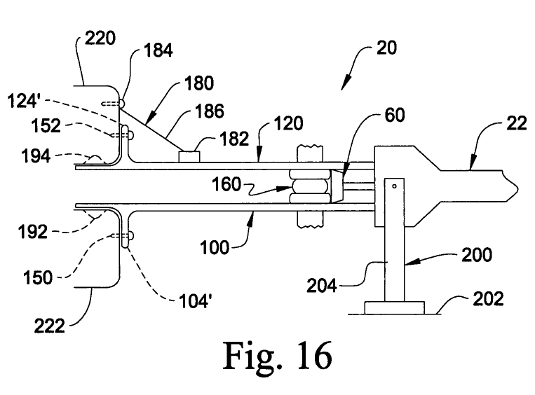

It is also contemplated that the inserter instrument 20 can be engaged to one or more of the vertebrae 220, 222 or other structure to maintain a positioning of inserter instrument 20 as the implant is positioned between the vertebrae and/or as one or more of the vertebrae 220, 222 are prepared to receive the implant with the chisel or other instrument. For example, as the implant is positioned between the vertebrae, the anchoring members 168, 170 or some other portion of the implant may meet resistance to distal movement upon contact with the bony structure of the vertebrae, and such resistance may tend to displace inserter instrument 20 proximally as increased force is applied to overcome the resistance. Various attachment arrangements are contemplated, examples of which are shown in FIG. 16, to maintain the positioning of inserter instrument 20 relative to the vertebrae and facilitate insertion of the implants and/or instruments.

In one arrangement, an external attachment arrangement 200 is provided that includes an attachment member 204 connectable to, for example, housing 22 and a support structure 202. Support structure 202 can be a surgical table, stand, wall, floor or other device that provides sufficient stability to maintain the positioning of inserter instrument 20 during the procedure. Attachment member 204 can be an arm, link, cable, bracket, support system, or other device that extends between and rigidly links inserter instrument 20 to support structure 202 at least when forces are applied that tend to displace inserter instrument 20 away from vertebrae 220. 222. Attachment member 204 could be attached to any suitable portion of inserter instrument 20.

In another arrangement, at least one of the support portions 107, 127 of guide members 100, 120 includes an attachment member 192, 194 that is positionable through the endplate of an adjacent one of the vertebrae 222, 220. Attachment members 192, 194 may be provided on one or both of the guide members 100, 120. Attachment members 192, 194 may comprise one or more spikes, teeth, ridges, or other structure that penetrates the respective adjacent vertebral endplate sufficiently to resist pull-out forces that might be encountered.

In another arrangement, at least one of the abutment members 104′, 124′ of guide members 100, 120 includes an attachment member 150, 152 that is positionable therethrough and engageable to an adjacent one of the vertebrae 222, 220. Attachment members 150, 152 may be provided on one or both of the guide members 100, 120. The respective abutment members 104′, 124′ may be provided with a hole to receive the attachment member, and may be provided with a sufficient height along the respective vertebra 222, 220 to allow placement and engagement of the respective attachment member 150, 152 therethrough. Attachment members 150, 152 may each comprise one or more bone screws, spikes, anchors, bolts, teeth, barbs, staples, suture anchor, suture, cable or other suitable attachment device that engages the respective adjacent vertebra sufficiently to resist pull-out forces that might be encountered.

In yet another embodiment, an attachment arrangement 180 includes a first securing member 182, a second securing member 184 and an attachment member 186 extending therebetween. Attachment arrangement 180 can be provided for one or both of the vertebrae 220, 222. The securing members 182, 184 provide a location for securing the attachment member 186 to the guide member 120 and vertebra 220, respectively. Securing members may be any type of fastener, block, or other member or location to which attachment member 186 can be engaged. Attachment member 186 can be a wire, cable, suture, cord, link, bar, strut or other device with sufficient tensile strength to resist pull-out forces that might be encountered.

The instruments discussed herein can protect the adjacent tissue and vasculature from the implant during insertion by preventing the implant 160 and spreader 60 from twisting and moving outside the guide path during insertion. The instruments further protect the bony structures between which the implant is inserted during insertion, and facilitate withdrawal of the implant after it is positioned in the space between the bony structures. Furthermore, the instruments can be adapted to guide insertion of implants of various heights, and to provide varying spacing between adjacent bony portions customized to fit the particular implant. The instruments include a low profile in the operative space, facilitating visualization and placement of additional instruments in the operative approach to the bony structures. The instruments are simple to disassemble, allowing for cleaning and use of selected guide members from a set of guide members, providing convenience and flexibility to the surgeon during the surgical procedure.

While the invention has been illustrated and described in detail in the drawings and foregoing description, the same is to be considered as illustrative and not restrictive in character. All changes and modifications that come within the spirit of the invention are desired to be protected.

What is claimed is:

1. An instrument for positioning an implant in a space between adjacent vertebrae, comprising:
a housing;
a pair of opposing guide members coupled to said housing, each of said pair of guide members including a body with an outer surface and an opposite guide surface and an elongated slot opening therebetween, said slot extending along said respective guide member and opening at a distal end thereof, said distal ends being positionable in the space between vertebrae;
a spreader positioned between said pair of guide members, said spreader including a central body and a pair of opposite wings extending therefrom, each wing including a body slidingly received in said slot of a corresponding one of said pair of guide members and an enlarged outer end; and
a drive member coupled to said spreader and operable to forwardly advance said spreader toward said distal ends of said guide members.

2. The instrument of claim 1, wherein said housing includes a coupling portion and a drive member engaging portion extending proximally from said coupling portion, said drive member engaging portion and said coupling portion including a passage extending therethrough for receiving said drive member.

3. The instrument of claim 2, wherein said drive member includes a shaft threadingly engaged in said passage and a handle at a proximal end of said shaft.

4. The instrument of claim 1, wherein said pair of guide members each include an abutment member adjacent said distal end thereof, said abutment member projecting from said outer surface of said respective guide member.

5. The instrument of claim 4, wherein each of said slots extends through said abutment member of said respective guide member.

6. The instrument of claim 5, wherein each of said guide members includes a support portion extending distally of said abutment member thereof and said slot of said respective guide member extends through and opens at a distal end of said support portion.

7. The instrument of claim 6, wherein said support portions are positionable in the space between the adjacent vertebrae with said abutment members in contact with respective ones of the adjacent vertebrae, said support portions being contactable with the adjacent vertebrae to distract the adjacent bony portions as said spreader is advanced toward said distal ends of said guide members.

8. The instrument of claim 1, wherein said spreader includes a pair of distally extending projections for engaging the implant between the guide members.

9. The instrument of claim 1, further comprising a chisel engageable with at least one of said guide members in interfitting relation, said chisel movable along said at least one guide member to cut at least one of the adjacent vertebrae when said distal ends of said guide members are positioned between the adjacent vertebrae.

10. The instrument of claim 9, wherein said at least one guide member includes a rail along each of opposite sides thereof, said rails each defining a groove for receiving an extension of said chisel.

11. The instrument of claim 9, wherein said chisel includes a central blade alignable along said slot of said at least one guide member.

12. The instrument of claim 11, wherein said chisel includes an abutment member at a proximal end of said blade extending laterally outwardly from said blade to contact the at least one vertebrae and limit insertion depth of said blade therein.

13. The instrument of claim 1, further comprising an attachment arrangement for securing the instrument to at least one of the adjacent vertebrae.

14. A system for stabilizing a spinal disc space while preserving motion capabilities of the vertebrae adjacent the disc space, comprising:
a pair of opposing guide members extending from a handle assembly, each of said pair of guide members including a body with an outer surface and an opposite guide surface and a spreader positioned between said pair of guide members;
an artificial disc implant including upper and lower plate members and an articulating member therebetween, said implant being positionable between said guide surfaces forwardly of and in engagement with said spreader with at least one anchoring member of the artificial disc implant extending through at least one of the opposing guide members; and
a drive member coupled to said spreader and operable to forwardly advance said spreader and said artificial disc implant toward distal ends of said guide members.

15. The system of claim 14, further comprising an engaging member extending between said spreader and each of said plate members to secure said plate members with said spreader.

16. The system of claim 14, each of said pair of guide members includes an elongated slot opening between said guide surface and said outer surface, said slot extending along said respective guide member and opening at a distal end thereof.

17. The system of claim 16, wherein said artificial disc include an anchoring member extending from each of said upper and lower plate members, said anchoring members extending through said elongated slot of an adjacent one of said guide members.

18. The system of claim 17, wherein said spreader includes a central body and a pair of opposite wings extending therefrom, each wing including a body slidingly received in said slot of a corresponding one of said pair of guide members and an enlarged outer end sized to prevent passage of said outer end through said slot.

19. The system of claim 14, further comprising a chisel engageable with at least one of said guide members in interfitting relation, said chisel movable along said at least one guide member to cut at least one of the adjacent vertebrae when said distal ends of said guide members are positioned in the disc space.

20. The system of claim 19, wherein said at least one guide member includes a rail along each of opposite sides thereof, said rails each defining a groove for receiving an extension of said chisel.

21. The system of claim 19, wherein said chisel includes a central blade alignable along said slot of said at least one guide member.

22. The system of claim 21, wherein said chisel includes an abutment member at a proximal end of said blade extending laterally outwardly from said blade to contact the at least one vertebrae and limit insertion depth of said blade therein.

23. The system of claim 14, wherein at least one of said guide members is pivotal relative to said housing away from the other of said guide members to permit placement of said artificial disc implant between said guide members, said artificial disc implant including anchoring members extending from each of said first and second plate members, said anchoring members being positioned through an elongated slot extending along an adjacent one of said guide members.

24. A method for inserting an implant in a space between adjacent vertebrae, comprising:
providing an implant inserter comprising:
a housing;
a pair of opposing guide members coupled to the housing, the guide members each including an elongate central slot extending therealong and opening at a distal end of the guide member;
a spreader positioned between the pair of guide members;
a drive member coupled to the spreader and extending though the housing;
pivoting at least one of the pair of guide members away from the other of the pair of guide members;
positioning an implant on the other of the pair of guide members and forwardly of the spreader, the implant including a first anchoring member extending through the slot of the other guide member; and
pivoting the at least one guide member toward the other guide member to receive a second anchoring member of the implant through the slot of the at least one guide member.

25. The method of claim 24, wherein providing the inserter includes providing the spreader with a central body and a pair of opposite wings extending therefrom each slidingly received in the slot of an adjacent one of the guide members.

26. The method of claim 25, further comprising:
positioning distal support portions of the guide members in the space;
manipulating the drive member to distally advance the spreader and implant between the guide members toward the space; and
distracting the adjacent vertebrae with the support portions as the distal advancement of the implant and spreader move the guide members away from one another.

27. The method of claim 26, further comprising distally advancing the implant into the space until the wings contact the adjacent vertebrae.

28. The method of claim 27, further comprising withdrawing the support portions from the space between the implant and the adjacent vertebrae by manipulating the drive member to push the wings against the adjacent vertebrae thereby proximally displacing the guide members relative to the vertebrae and the implant and passing the anchoring members through the distal end openings of the slots of the guide members.

29. The method of claim 24, wherein before positioning the implant:
positioning distal support portions of the guide members in the space;
manipulating the drive member to distally advance the spreader between the guide members toward the space;
distracting the adjacent vertebrae with the support portions as the distal advancement of the implant and spreader move the guide members away from one another; and
guiding a chisel along at least one of the guide member and cutting a path in at least one of the adjacent vertebrae.

30. The method of claim 29, wherein the chisel includes an abutment member distal of a blade of the chisel, the abutment member contacting the at least one vertebra to limit an insertion depth of the blade into the at least one vertebra.

31. The method of claim 30, further comprising:
removing the chisel from the guide member before positioning the implant; and
positioning distal support portions of the guide members in the space;
manipulating the drive member to distally advance the spreader and implant between the guide members toward the space; and
distracting the adjacent vertebrae with the support portions as the distal advancement of the implant and spreader move the guide members away from one another.

32. The method of claim 31, further comprising distally advancing the implant into the space, the implant including at least one anchoring member extending through the guide member into the path in the at least one vertebra.
